(19) United States
(12) Patent Application Publication
(10) Pub. No.: US 2007/0081982 A1
(43) Pub. Date: Apr. 12, 2007

(54) Multiple RNAi expression cassettes for simultaneous delivery of RNAi agents related to heterozygotic expression patterns

(76) Inventors: Elisabeth Evertsz, San Jose, CA (US); Sarah Brashears, Mountain View, CA (US)

Correspondence Address: PATTERSON & SHERIDAN, L.L.P., 3040 POST OAK BOULEVARD, SUITE 1500, HOUSTON, TX, 77056, US

(21) Appl. No.: 11/413,628

(22) Filed: Apr. 28, 2006

Related U.S. Application Data
(60) Provisional application No. 60/676,206, filed on Apr. 28, 2005.

(51) Int. Cl. A61K 48/00; C12N 5/08

(52) U.S. Cl. 424/93.21; 514/44; 435/366

(57) ABSTRACT

The present invention provides compositions and methods suitable for expressing y-x multiple-RNAi agents against an allele or alleles of interest in cells, tissues or organs of interest in vitro and in vivo so as to treat diseases.

### CROSS-REFERENCE TO RELATED APPLICATIONS

This application claims benefit of U.S. provisional patent application Ser. No. 60/676,206, filed Apr. 28, 2005, which is herein incorporated by reference.

### BACKGROUND OF THE INVENTION

Utilization of double-stranded RNA to inhibit gene expression in a sequence-specific manner has revolutionized the drug discovery industry. In mammals, RNA interference, or RNAi, is mediated by 15- to 49-nucleotide long, double-stranded RNA molecules referred to as small interfering RNAs (RNAi agents). RNAi agents can be synthesized chemically or enzymatically outside of cells and subsequently delivered to cells (see, e.g., Fire, et al., Nature, 391:806-11 (1998); Tuschl, et al., Genes and Dev., 13:3191-97 (1999); and Elbashir, et al., Nature, 411:494-498 (2001)); or can be expressed in vivo by an appropriate vector in cells (see, e.g., U.S. Pat. No. 6,573,099).

In vivo delivery of unmodified RNAi agents as an effective therapeutic for use in humans faces a number of technical hurdles. First, due to cellular and serum nucleases, the half life of RNA injected in vivo is only about 70 seconds (see, e.g., Kurreck, Eur. J. Bioch. 270:1628-44 (2003)). Efforts have been made to increase stability of injected RNA by the use of chemical modifications; however, there are several instances where chemical alterations led to increased cytotoxic effects. In one specific example, cells were intolerant to doses of an RNAi duplex in which every second phosphate was replaced by phosphorothioate (Harborth, et al., Antisense Nucleic Acid Drug Rev. 13(2): 83-105 (2003)). Still ongoing efforts are directed to find ways to delivery unmodified or modified RNAi agents so as to provide tissue-specific delivery, as well as deliver the RNAi agents in amounts sufficient to elicit a therapeutic response but that are not toxic.

Other options being explored for RNAi delivery include the use of viral-based and non-viral based vector systems that can infect or otherwise transfect target cells, and deliver and express RNAi molecules in situ. Often, small RNAs are transcribed as short hairpin RNA (shRNA) precursors from a viral or non-viral vector backbone. Once transcribed, the shRNA are processed by the enzyme Dicer into the appropriate active RNAi agents. Viral-based delivery approaches attempt to exploit the targeting properties of viruses to generate tissue specificity and once appropriately targeted, rely upon the endogenous cellular machinery to generate sufficient levels of the RNAi agents to achieve a therapeutically effective dose.

One useful application of RNAi therapeutics is in the treatment of disease caused by the differential expression of genes in a heterozygotic allelic pair. Over 1200 human disease genes have been discovered in the past two decades. Some examples of these diseases include breast cancer, Type 1 diabetes mellitus, epidermolysis bullosa simplex, lactose intolerance, cystic fibrosis, Fanconi anemia, and Alzheimer's. The genes associated with these diseases have been implicated in Mendelian and more genetically complex phenotypes.

The mutations in genes causing diseases can often be localized to a single nucleotide polymorphism (SNP) or group of SNPs known as a haplotype group. SNPs can arise in several ways. A single nucleotide polymorphism may arise due to a substitution of one nucleotide for another at the polymorphic site. Substitutions can be transitions or transversions. A transition is the replacement of one purine nucleotide by another purine nucleotide or one pyrimidine nucleotide by another pyrimidine nucleotide. A transversion is the replacement of a purine by a pyrimidine, or the converse.

Single nucleotide polymorphisms can also arise from a deletion of a nucleotide or an insertion of a nucleotide relative to a reference allele. Thus, a polymorphic site is a site at which one allele bears a gap with respect to a single nucleotide in another allele. Some SNPs occur within genes or near genes. One such class includes SNPs falling within regions of genes encoding for a polypeptide product. These SNPs may result in an alteration of the amino acid sequence of the polypeptide product and give rise to the expression of a defective or other variant protein. Such variant products can, in some cases, result in a pathological condition, e.g., genetic disease. Examples of diseases in which a polymorphism within a coding sequence gives rise to genetic disease include Hypercholesterolemia, Marfans and epidermolysis bullosa simplex. These diseases are classified as autosomal dominant diseases, because a defect in one allele of a pair results in the disease phenotype. Selective decrease in the expression product of the disease allele can result in the reduction of disease phenotype. Thus, there is a need in the art to develop stable, effective RNAi methods to specifically alter the expression of a disease allele.

### SUMMARY OF THE INVENTION

The present invention provides stable, effective ddRNAi reagents and methods for use thereof to control the expression of disease genes by altering the level of expression of one or more transcriptionally active genetic regions of only one allele of a heterozygotic allele pair.

The present invention provides a method for allele-specific control of genes together with genetic agents for use therewith, as well as genetically modified cells comprising the genetic agents. The present invention targets one or two or more polymorphic targets in a single gene or multiple genes in order to modify the expression of one or more alleles in a heterozygotic gene pair or group of gene pairs. The present invention allows for changes in the expression of one or two or more genes containing SNPs or other polymorphisms that relate to disease without altering the expression of alleles expressing the normal or wild type version of the gene. In embodiments where only one region receives silencing, multiple-RNAi constructs are used to target multiple SNPs in a haplotype group. In embodiments where more than one genetic region must be silenced, the present invention provides the use of genetic agents that facilitate gene silencing via multiple-RNAi constructs to down regulate or silence one or more transcriptionally active genetic regions of a particular allele in a heterozygotic allelic pair that is directly or indirectly associated with disease. Such multiple RNAi constructs may have one promoter or multiple promoters. Such transcriptionally active regions are also referred to herein as “single nucleotide genetic targets” or “SNTs”. ddRNAi-mediated silencing of one or more SNTs effects control of the one allele of a heterozygotic pair in a subject or cell culture. RNAi agents of this invention can be specific for one or two or more allelic variants of a disease gene while not significantly impacting the expression of the normal allele.

Accordingly, one aspect of the present invention provides a method for affecting gene expression of one or more genes in a subject or cell culture, said method comprising administering to said subject or cell culture a genetic construct comprising at least one ddRNAi expression cassette which encodes an RNA molecule comprising one, two or multiple RNAi nucleotide sequences which are individually at least 90% identical to at least part of a nucleotide sequence comprising one or more single nucleotide genetic targets (SNTs) or derivatives, orthologs or homologs thereof and which delay, repress or otherwise reduce the expression of one or more SNTs in said subject or cell culture while not affecting the expression of the normal allele of the heterozygotic pair. The multiple-RNAi constructs of the instant invention are designated by y-x nomenclature designating the number of promoters (y) and the number of RNAi agents (x). The y-x constructs of this invention are comprised of two or more RNAi sequences under the control of a single promoter generating a single promoter/multiple RNAi construct (1-x RNAi construct) or a construct comprised of two or more promoters each controlling a single RNAi construct generating a multiple promoter/multiple RNAi construct (y-x) RNAi construct.

In another aspect, the present invention provides genetically modified cells comprising a ddRNAi expression construct as described herein. Preferably the cell is a mammalian cell, even more preferably the cell is a primate or rodent cell and most preferably the cell is a human or mouse cell. Furthermore, in yet another aspect, the present invention provides a multi-cellular structure comprising one or more genetically modified cells of the present invention. Multi-cellular structures include, inter alia, a tissue, organ or complete organism.

Other objects and advantages of the present invention will be apparent from the detailed description that follows.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIG. 1 is a simplified block diagram of one embodiment of a method for delivering RNAi species according to the present invention.

FIGS. 2A and 2B are simplified schematic representations of embodiments of a multiple-promoter/multiple-RNAi expression cassette of the present invention.

FIGS. 3A and 3B show two embodiments of multiple-promoter/multiple-RNAi expression cassettes that deliver RNAi agents as shRNA precursors. FIG. 3C shows an embodiment of a multiple-RNAi expression cassette comprising stuffer regions inserted between promoter/RNAi/terminator components. FIGS. 3D and 3E show embodiments of multiple-RNAi expression cassettes that deliver RNAi without a shRNA precursor.

FIG. 4A and 4B are simplified schematic representations of embodiments of a single-promoter/multiple RNAi expression cassette of the present invention.

FIG. 5A and 5B are simplified representations of methods of producing multiple-RNAi expression vectors packaged in viral particles.

### DETAILED DESCRIPTION

Before the present compositions and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

As used herein, the singular forms “a,” “and,” and “the” include plural referents unless the context clearly dictates otherwise. Thus, for example, reference to “a factor” refers to one or mixtures of factors, and reference to “the method of production” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference, without limitation, for the purpose of describing and disclosing devices, formulations and methodologies which are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

The present invention is directed to innovative, robust genetic compositions and methods to deliver at least three different RNAi agents simultaneously to a cell using a single expression construct. The compositions and methods provide stable, lasting inhibition of target nucleic acids.

Generally, conventional methods of molecular biology, microbiology, recombinant DNA techniques, cell biology, and virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A practical Approach, (Alan, J. Cann, Ed., Oxford University Press, 2000).

A “vector” is a replicon, such as plasmid, phage, viral construct or cosmid, to which another DNA segment may be attached. Vectors are used to transduce and express the DNA segment in cells.

A “promoter” or “promoter sequence” is a DNA regulatory region capable of binding RNA polymerase in a cell and initiating transcription of a polynucleotide or polypeptide coding sequence such as messenger RNA, ribosomal RNAs, small nuclear of nucleolar RNAs or any kind of RNA transcribed by any class of any RNA polymerase I, II or III.

A cell has been “transformed”, “transduced” or “transfected” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell. With respect to eukaryotic cells, a stably transformed cell is one in which the transforming DNA has become integrated into a host cell chromosome or is maintained extra-chromosomally so that the transforming DNA is inherited by daughter cells during cell replication or is a non-replicating, differentiated cell in which a persistent episome is present.

The term “RNA interference” or “RNAi” refers generally to a process in which a double-stranded RNA molecule or a short hairpin RNA changes the expression of a nucleic acid sequence with which they share substantial or total homology. The term “RNA species” or “RNAi agent” refers to a distinct RNA sequence that elicits RNAi; and the term “RNAi expression cassette” refers to a cassette according to embodiments of the present invention comprising three or more RNAi species.

The term ‘multiple-RNAi constructs’ of the instant invention use the following nomenclature to designate the number of promoters (y) and the number of RNAi agents (x). In one embodiment of the invention the y-x RNAi constructs of this invention are comprised of two or more RNAi sequence under the control of a single promoter (1-x). A construct comprising two promoters each controlling an RNAi construct respectively is a 2-2 multiple promoter/multiple RNAi construct and so on. y number of promoters can be two or three or more. x, the number of RNAi agents can be two or three or more. Preferably y is less than or equal to x.

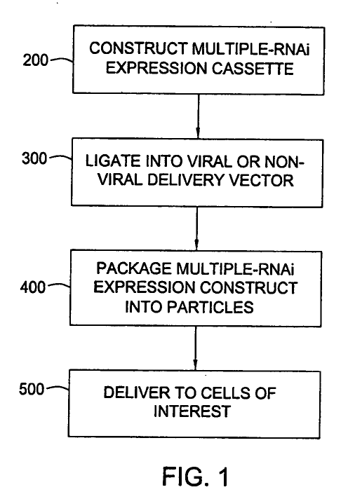

FIG. 1 is a simplified flow chart showing the steps of one method in which the multiple-RNAi expression constructs according to the present invention may be used. First, in step 200, a multiple-RNAi expression cassette targeting a particular disease target is constructed. Next, in step 300, the multiple-RNAi expression cassette is ligated into an appropriate viral or non viral delivery construct. The RNAi expression delivery construct is then packaged into viral particles at step 400, and the viral particles are delivered to the target cells to be treated at step 500. Details for each of these steps and the components involved are presented infra.

The multiple-RNAi expression constructs according to the present invention can be generated synthetically or enzymatically by a number of different protocols known to those of skill in the art and purified using standard recombinant DNA techniques as described in, for example, Sambrook et al., Molecular Cloning: A Laboratory Manual, 2nd Ed., Cold Spring Harbor Press, Cold Spring Harbor, N.Y. (1989), and under regulations described in, e.g., United States Dept. of HHS, National Institute of Health (NIH) Guidelines for Recombinant DNA Research. In a preferred embodiment, the multiple-RNAi expression cassettes are synthesized using phosphoramidite, or analogous chemistry using protocols well known in the art.

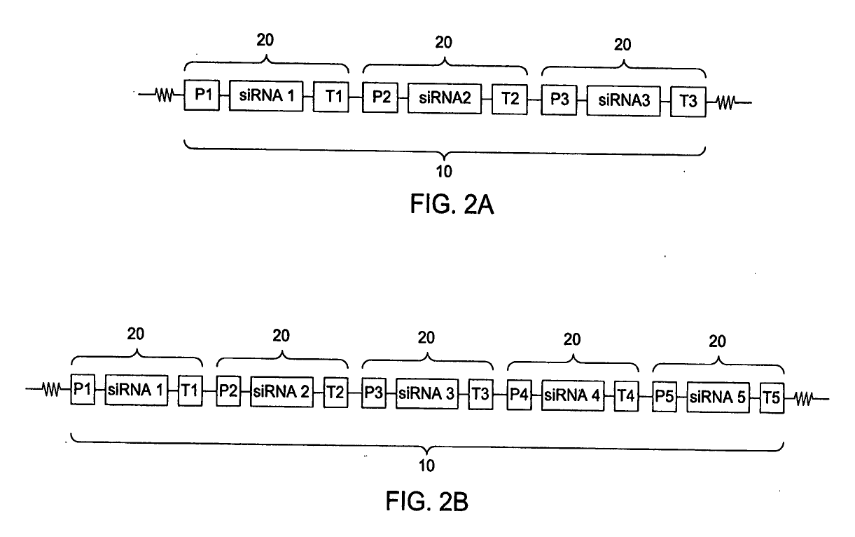

FIG. 2A and 2B are simplified schematics of multiple-RNAi expression constructs according to embodiments of the present invention. FIG. 2A shows an embodiment of a multiple-RNAi expression cassette (10) with three promoter/RNAi/terminator components (3-3 RNAi expression cassette), and FIG. 2B shows an embodiment of a multiple-promoter expression cassette (10) with five promoter/RNAi/terminator components (5-5 RNAi expression cassette). P1, P2, P3, P4 and P5 represent promoter elements. RNAi1, RNAi2, RNAi3, RNAi4 and RNAi5 represent sequences for five different RNAi species. T1, T2, T3, T4, and T5 represent termination elements. The multiple-RNAi expression cassettes according to the present invention may contain three or more promoter/RNAi/terminator components where the number of promoter/RNAi/ terminator components included in any multiple-promoter RNAi expression cassette is limited by, e.g., packaging size of the delivery system chosen (for example, some viruses, such as AAV, have relatively strict size limitations); cell toxicity, and maximum effectiveness (i.e. when, for example, expression of four RNAi sequences is as effective therapeutically as the expression of ten RNAi sequences).

The three or more RNAi species in the multiple-RNAi components comprising a cassette all have different sequences; that is RNAi1, RNAi2, RNAi3, RNAi4 and RNAi5 are all different from one another. However, the promoter elements in any cassette may be the same (that is, e.g., the sequence of two or more of P1, P2, P3, P4 and P5 may be the same); all the promoters within any cassette may be different from one another; or there may be a combination of promoter elements represented only once and promoter elements represented two times or more within any cassette. Similarly, the termination elements in any cassette may be the same (that is, e.g., the sequence of two or more of T1, T2, T3, T4 and T5 may be the same, such as contiguous stretches of 4 or more T residues); all the termination elements within any cassette may be different from one another; or there may be a combination of termination elements represented only once and termination elements represented two times or more within any cassette. Preferably, the promoter elements and termination elements in each promoter/RNAi/terminator component comprising any cassette are all different to decrease the likelihood of DNA recombination events between components and/or cassettes. Further, in a preferred embodiment, the promoter element and termination element used in each promoter/RNAi/terminator component are matched to each other; that is, the promoter and terminator elements are taken from the same gene in which they occur naturally.

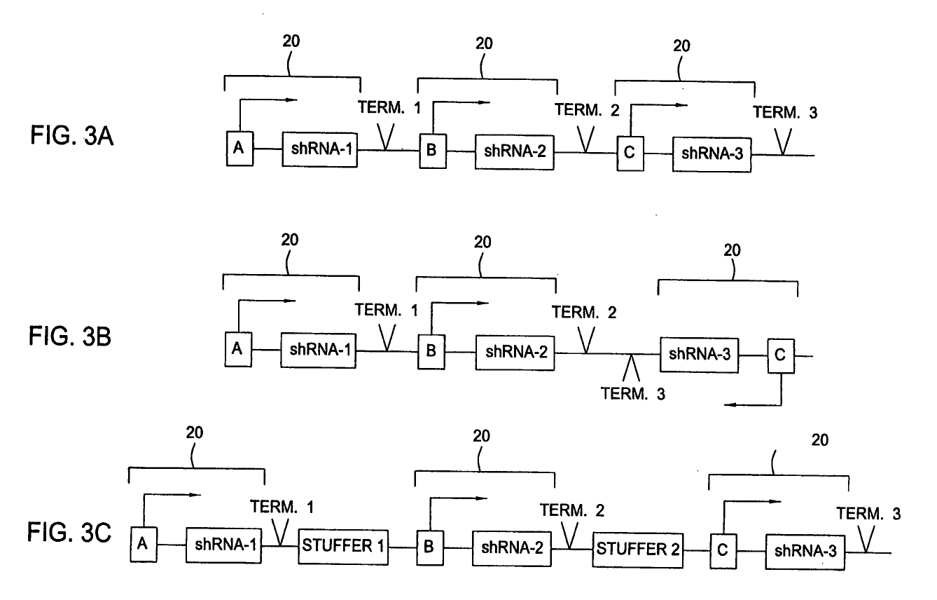

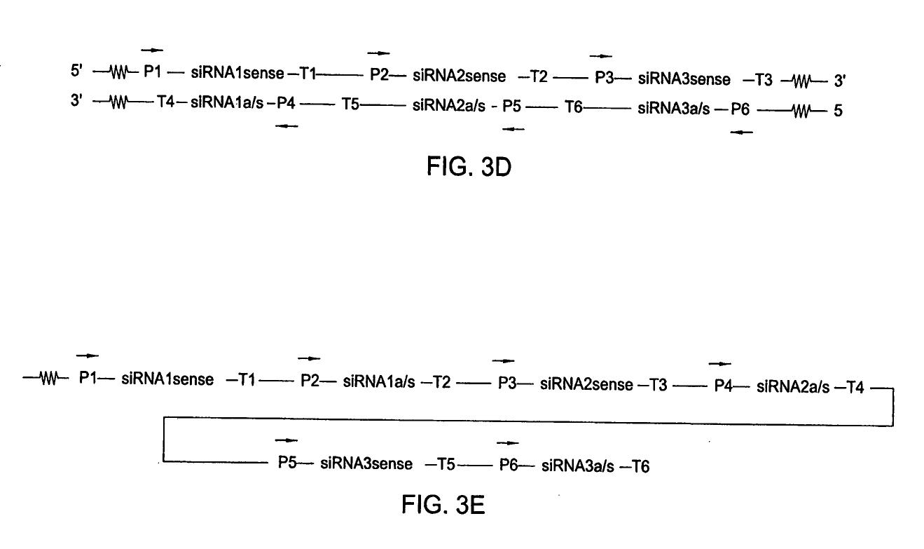

FIGS. 3A, 3B and 3C show multiple-RNAi expression constructs comprising alternative embodiments of multiple-RNAi expression cassettes that express short shRNAs. shRNAs are short duplexes where the sense and antisense strands are linked by a hairpin loop. Once expressed, shRNAs are processed into RNAi species. Boxes A, B and C represent three different promoter elements, and the arrows indicate the direction of transcription. TERM 1, TERM 2, and TERM 3 represent three different termination sequences, and shRNA-1, shRNA-2 and shRNA-3 represent three different shRNA species. The multiple-RNAi expression cassettes in the embodiments extend from the box marked A to the arrow marked Term3. FIG. 3A shows each of the three promoter/RNAi/terminator components (20) in the same orientation within the cassette, while FIG. 3B shows the promoter/RNAi/terminator components for shRNA-1 and shRNA-2 in one orientation, and the promoter/RNAi/terminator component for shRNA-3 in the opposite orientation (i.e., transcription takes place on both strands of the cassette).

FIG. 3C shows each of the cassettes separated by a region of DNA to increase the distance between promoter/RNAi/terminator components. The inserted DNA, known as “stuffer” DNA, can be any length between 5-5000 nucleotides. There can be one or more stuffer fragments between promoters. In the case of multiple stuffer fragments, they can be the same or different lengths. The stuffer DNA fragments are preferably different sequences. The stuffer DNA fragments may be used to increase the size of the multiple-RNAi cassette of the present invention in order to allow it to fit appropriately into a corresponding delivery vector. The length of the stuffer is dictated by the size requirements of the particular vector associated with the multiple-RNAi cassette. For example, in one embodiment the stuffer fragments total 4000 nucleotides (nt) in order to appropriately fulfill the size requirements of the AAV vector. In another embodiment, the stuffer fragments total 2000 nt in order to appropriately fulfill the size requirements of the self complementary AAV vector. Other variations may be used as well.

FIGS. 3D and 3E show multiple-RNAi expression constructs comprising alternative embodiments of multiple-promoter RNAi expression cassettes that express RNAi species without a hairpin loop. In both figures, P1, P2, P3, P4, P5 and P6 represent promoter elements (with arrows indicating the direction of transcription); and T1, T2, T3, T4, T5, and T6 represent termination elements. Also in both figures, RNAi1 sense and RNAi1 antisense (a/s) are complements, RNAi2 sense and RNAi2 a/s are complements, and RNAi3 sense and RNAi3 a/s are complements.

In the embodiment shown in FIG. 3D, all three RNAi sense sequences are transcribed from one strand (via P1, P2 and P3), while the three RNAi a/s sequences are transcribed from the complementary strand (via P4, P5, P6). In this particular embodiment, the termination element of RNAi1 a/s (T4) falls between promoter P1 and the RNAi 1 sense sequence; while the termination element of RNAi1 sense (T1) falls between the RNAi1 a/s sequence and its promoter, P4. This motif is repeated such that if the top strand shown in FIG. 3D is designated the (+) strand and the bottom strand is designated the (−) strand, the elements encountered moving from left to right would be P1(+), T4(−), RNAi1 (sense and a/s), T1(+), P4(−), P2(+), T5(−), RNAi2 (sense and a/s), T2(+), P5(−), P3(+), T6(−), RNAi3 (sense and a/s), T3(+), and P6(−).

In an alternative embodiment shown in FIG. 3E, all RNAi sense and antisense sequences are transcribed from the same strand. One skilled in the art appreciates that any of the embodiments of the multiple-promoter RNAi expression cassettes shown in FIGS. 3A through 3E may be used for certain applications, as well as combinations or variations thereof.

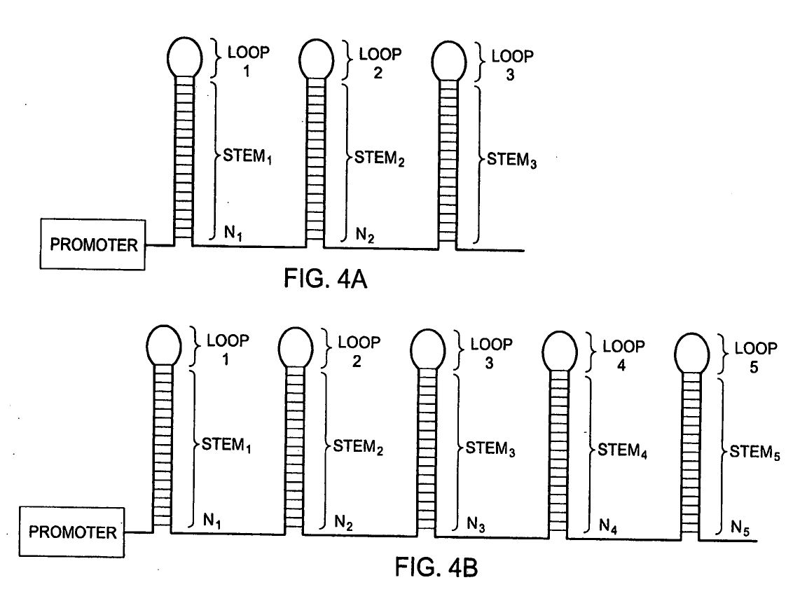

FIGS. 4A and 4B are simplified schematics of 1-3 and 1-5 RNAi expression cassettes according to embodiments of the present invention containing three and five RNAi stem-loop structures respectively. It should be understood by those skilled in the art that 1-x RNAi expression cassettes of the present invention may contain two, four, six or more stem-loop structures and that the embodiments shown in this figure are exemplary. The figures show embodiments of the 1-3 and 1-5 RNAi expression cassettes comprising one promoter and three or five stem-loop structures separated by spacer regions. The stem regions 1-5 comprise between about 17-21 base pairs, preferably 19 base pairs. The loop regions 1-5 comprise between about 3-20 nucleotides, preferably 5 to 9 nucleotides, more preferably 6 nucleotides. The spacer regions (N₁, N₂ . . . ) between RNAi stems are between about 4-10 nucleotides, preferably 6 nucleotides.

In some embodiments, promoters of variable strength may be employed. For example, use of three or more strong promoters (such as a Pol III-type promoter) may tax the cell under some circumstances, by, e.g., depleting the pool of available nucleotides or other cellular components needed for transcription. In addition or alternatively, use of several strong promoters may cause a toxic level of expression of RNAi agents in the cell. Thus, in some embodiments one or more of the promoters in the multiple-promoter RNAi expression cassette may be weaker than other promoters in the cassette, or all promoters in the cassette may express RNAi agents at less than a maximum rate. Promoters also may or may not be modified using molecular techniques, or otherwise, e.g., through regulation elements, to attain weaker levels of transcription.

Promoters may be tissue-specific or cell-specific. The term “tissue specific” as it applies to a promoter refers to a promoter that is capable of directing selective expression of a nucleotide sequence of interest to a specific type of tissue (e.g., liver) in the relative absence of expression of the same nucleotide sequence of interest in a different type of tissue (e.g., brain). Such tissue specific promoters include promoters such as Ick, myogenin, or thy1. The term “cell-specific” as applied to a promoter refers to a promoter which is capable of directing selective expression of a nucleotide sequence of interest in a specific type of cell in the relative absence of expression of the same nucleotide sequence of interest in a different type of cell within the same tissue (see, e.g., Higashibata, et al., J. Bone Miner. Res. January 19(1):78-88 (2004); Hoggatt, et al., Circ. Res., December 91(12):1151-59 (2002); Sohal, et al., Circ. Res. July 89(1):20-25 (2001); and Zhang, et al., Genome Res. January 14(1):79-89 (2004)). The term “cell-specific” when applied to a promoter also means a promoter capable of promoting selective expression of a nucleotide sequence of interest in a region within a single tissue. Alternatively, promoters may be constitutive or regulatable. Additionally, promoters may be modified so as to possess different specificities.

The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a coding sequence in substantially any cell and any tissue. The promoters used to transcribe the RNAi species preferably are constitutive promoters, such as the promoters for ubiquitin, CMV, β-actin, histone H4, EF-1alfa or pgk genes controlled by RNA polymerase II, or promoter elements controlled by RNA polymerase I. In preferred embodiments, promoter elements controlled by RNA polymerase III are used, such as the U6 promoters (U6-1, U6-8, U6-9, e.g.), H1 promoter, 7SL promoter, the human Y promoters (hY1, hY3, hY4 (see Maraia, et al., Nucleic Acids Res 22(15):3045-52 (1994)) and hY5 (see Maraia, et al., Nucleic Acids Res 24(18):3552-59 (1994)), the human MRP-7-2 promoter, Adenovirus VA1 promoter, human tRNA promoters, the 5s ribosomal RNA promoters, as well as functional hybrids and combinations of any of these promoters.

Alternatively in some embodiments it may be optimal to select promoters that allow for inducible expression of the RNAi species. A number of systems for the inducible expression using such promoters are known in the art, including but not limited to the tetracycline responsive system and the lac operator-repressor system (see PCT publication WO 03/022052 A1; and U.S. Patent Application Publication No. 2002/0162126 A1), the ecdysone regulated system, or promoters regulated by glucocorticoids, progestins, estrogen, RU-486, steroids, thyroid hormones, cyclic AMP, cytokines, the calciferol family of regulators, or the metallothionein promoter (regulated by inorganic metals).

One or more enhancers also may be present in the multiple-RNAi expression constructs of the present invention to increase expression of the gene of interest. Enhancers appropriate for use in embodiments of the present invention include the Apo E HCR enhancer, the CMV enhancer that has been described recently (see, Xia et al, Nucleic Acids Res 31-17 (2003)) if expression in liver is desired, and other enhancers known to those skilled in the art, for example a myocyte-specific enhancer that directs transcription in muscle cells.

The RNAi sequences encoded by the RNAi expression cassettes of the present invention result in the expression of one or more small interfering RNAs that are short, double-stranded RNAs that are not toxic in mammalian cells. There is no particular limitation in the length of the RNAi species of the present invention as long as they do not show cellular toxicity. RNAis can be, for example, 15 to 49 bp in length, preferably 15 to 35 bp in length, and are more preferably 19 to 29 bp in length. The double-stranded RNA portions of RNAis may be completely homologous, or may contain non-paired portions due to sequence mismatch (the corresponding nucleotides on each strand are not complementary), bulge (lack of a corresponding complementary nucleotide on one strand), and the like. Such non-paired portions can be tolerated to the extent that they do not significantly interfere with RNAi duplex formation or efficacy.

The termini of an RNAi species according to the present invention may be blunt or cohesive (overhanging) as long as the RNAi effectively silences the target gene. The cohesive (overhanging) end structure is not limited only to a 3′ overhang, but a 5′ overhanging structure may be included as long as the resulting RNAi is capable of inducing the RNAi effect. In addition, the number of overhanging nucleotides may be any number as long as the resulting RNAi is capable of inducing the RNAi effect. For example, if present, the overhang may consist of 1 to 8 nucleotides; preferably it consists of 2 to 4 nucleotides.

The RNAi species utilized in the present invention may have a stem-loop structured precursor (shRNA) in which the ends of the double-stranded RNA are connected by a single-stranded, linker RNA. The length of the single-stranded loop portion of the shRNA may be 5 to 20 bp in length, and is preferably 5 to 9 bp in length.

Any transcribed nucleic acid sequence associated with an autosomal dominant disease may be a target for the RNAi agents of the present invention. Likely targets for the RNAi are genes such as but not limited to developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); viral structural genes such as capsid and envelope proteins; bacterial genes such as those involved in replication or structural features, or genes from other pathogens that are involved in replication or structural features. In addition, the multiple RNAi expression cassettes of the present invention may be used to target specific sequences that are unique to alleles responsible for pathology in autosomal dominant diseases such as SCA, the allele responsible for Huntington's disease, or the collagen gene alleles responsible for osteogenesis imperfecta.

An important aspect of the present invention is that individual disease causing alleles of targeted genes can be silenced by siRNA agents and result in no or reduced effect on the expression of the normal allele. Another aspect of this invention is that multiple SNPs of one or more specific alleles can be simultaneously targeted by the multiple RNAi agents of this invention. This feature of the present invention distinguishes it from prior art methods, where only a single aspect of a single gene can be targeted.

Methods of alignment of sequences for comparison and RNAi sequence selection are well known in the art. The determination of percent identity between two or more sequences can be accomplished using a mathematical algorithm. Preferred, non-limiting examples of such mathematical algorithms are the algorithm of Myers and Miller (1988); the search-for-similarity-method of Pearson and Lipman (1988); and that of Karlin and Altschul (1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing-BLAST analyses is publicly available through the National Center for Biotechnology Information (http://www.ncbi.nlm.nih.gov/).

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Typically, inhibition of target sequences by RNAi requires a high degree of sequence homology between the target sequence and the sense strand of the RNAi molecules. In some embodiments, such homology is higher than about 70%, and may be higher than about 75%. Preferably, homology is higher than about 80%, and is higher than 85% or even 90%. More preferably, sequence homology between the target sequence and the sense strand of the RNAi is higher than about 90%, 91%, 92%, 93%, 94%, 95%, 96%, 97%, 98% or 99%.

The multiple-RNAi expression constructs of the present invention are particularly useful when targeting heterozygous haplotype groups that are specific for a particular allele.

In addition to selecting the RNAi sequences based on conserved regions of a target sequence, selection of the RNAi sequences may be based on other factors. Despite a number of attempts to devise selection criteria for identifying sequences that will be effective in RNAi based on features of the desired target sequence (e.g., percent GC content, position from the translation start codon, or sequence similarities based on an in silico sequence database search for homologs of the proposed RNAi, thermodynamic pairing criteria), it is presently not possible to predict with much degree of confidence which of the myriad possible candidate RNAi sequences correspond to a desired target will, in fact, elicit an RNA silencing response. Instead, individual specific candidate RNAi polynucleotide sequences typically are generated and tested to determine whether interference with expression of a desired target can be elicited.

As stated, the RNAi coding regions of the multiple RNAi expression cassettes are operatively linked to terminator elements. In one embodiment, the terminators comprise stretches of four or more thymidine residues. In one embodiment there is only one terminator. In another preferred embodiment, there is a separate terminator element for each promoter/RNAi element. In one embodiment, the terminator elements used are all different and are matched to the promoter elements from the gene from which the terminator is derived. Such terminators include the SV40 poly A, the Ad VA1 gene, the 5S ribosomal RNA gene, and the terminators for human t-RNAs. In addition, promoters and terminators may be mixed and matched, as is commonly done with RNA pol II promoters and terminators.

In addition, the transcribed RNAi agents may be configured where multiple cloning sites and/or unique restriction sites are located strategically, such that promoter, RNAi and terminator elements are easily removed or replaced. Moreover, the multiple-promoter RNAi expression cassettes may be assembled from smaller oligonucleotide components using strategically located restriction sites and/or complementary sticky ends. The base vector for one approach according to embodiments of the present invention consists of plasmid with a multilinker in which all sites are unique (though this is not an absolute requirement). Sequentially, each promoter is inserted between its designated unique sites resulting in a base cassette with three promoters, or more, all of which can have variable orientation. Sequentially, again, annealed primer pairs are inserted into the unique sites downstream of each of the individual promoters, resulting in a triple expression cassette construct. The insert can be moved into, e.g. an AAV backbone using two unique enzyme sites (the same or different ones) that flank the triple expression cassette insert.

In step 300 of FIG. 1, the multiple-RNAi expression cassettes are ligated into a delivery vector. The constructs into which the multiple-RNAi expression cassette is inserted and used for high efficiency transduction and expression of the RNAi agents in various cell types preferably are derived from viruses and are compatible with viral delivery. Generation of the construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. The construct preferably comprises, for example, sequences necessary to package the multiple-promoter RNAi expression construct into viral particles and/or sequences that allow integration of the multiple promoter RNAi expression construct into the target cell genome. The viral construct also may contain genes that allow for replication and propagation of virus, though in preferred embodiments such genes will be supplied in trans. Additionally, the viral construct may contain genes or genetic sequences from the genome of any known organism incorporated in native form or modified. For example, the preferred viral construct comprises sequences useful for replication of the construct in bacteria.

The construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments of the present invention include those coding for proteins which confer a selective growth advantage on cells such as adenosine deaminase, aminoglycodic phosphotransferase, dihydrofolate reductase, hygromycin-B-phosphotransferase, or those coding for proteins that provide a biosynthetic capability missing from an auxotroph. If a reporter gene is included along with the multiple-promoter RNAi expression cassette, an internal ribosomal entry site (IRES) sequence can be included. Preferably, the additional genetic elements are operably linked with and controlled by an independent promoter/enhancer.

A viral delivery system based on any appropriate virus may be used to deliver the multiple-promoter RNAi expression constructs of the present invention. In addition, hybrid viral systems may be of use. The choice of viral delivery system will depend on various parameters, such as the tissue targeted for delivery, transduction efficiency of the system, pathogenicity, immunological and toxicity concerns, and the like. Given the diversity of disease targets that are amenable to interference by the multiple-promoter RNAi expression constructs of the present invention, it is clear that there is no single viral system that is suitable for all applications. When selecting a viral delivery system to use in the present invention, it is important to choose a system where multiple-promoter RNAi expression construct-containing viral particles are preferably: 1) reproducibly and stably propagated; 2) able to be purified to high titers; and 3) able to mediate targeted delivery (delivery of the multiple-promoter RNAi expression construct to the tissue or organ of interest without widespread dissemination); 4) able to be expressed in a constitutive or regulatable manner.

In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses). This distinction is an important determinant of the suitability of each vector for particular applications; non-integrating vectors can, under certain circumstances, mediate persistent gene expression in non-proliferating cells, but integrating vectors are the tools of choice if stable genetic alteration needs to be maintained in dividing cells.

For example, in one embodiment of the present invention, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells.

Once in the nucleus, the virus uncoats and the transgene is expressed from a number of different forms—the most persistent of which are circular monomers. AAV will integrate into the genome of 1-5% of cells that are stably transduced (Nakai, et al., J. Virol. 76:11343-349 (2002). Expression of the transgene can be exceptionally stable and in one study with AAV delivery of Factor IX, a dog model continues to express therapeutic levels of the protein over 5.0 years after a single direct infusion with the virus. Because progeny virus is not produced from AAV infection in the absence of helper virus, the extent of transduction is restricted only to the initial cells that are infected with the virus. It is this feature which makes AAV a preferred gene therapy vector for the present invention. Furthermore, unlike retrovirus, adenovirus, and herpes simplex virus, AAV appears to lack human pathogenicity and toxicity (Kay, et al., Nature. 424: 251 (2003) and Thomas, et al., Nature Reviews, Genetics 4:346-58 (2003)). Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as RNAi.

Another viral delivery system useful with the multiple-promoter RNAi expression constructs of the present invention is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some embodiments, lentiviruses are the preferred members of the retrovirus family for use in the present invention. Lentivirus vectors are often pseudotyped with vesicular steatites virus glycoprotein (VSV-G), and have been derived from the human immunodeficiency virus (HIV), the etiologic agent of the human acquired immunodeficiency syndrome (AIDS); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization. The main advantage to the use of lentiviral vectors is that gene transfer is persistent in most tissues or cell types.

A lentiviral-based construct used to express the RNAi agents preferably comprises sequences from the 5′ and 3′ long terminal repeats (LTRs) of a lentivirus. More preferably the viral construct comprises an inactivated or self-inactivating 3′ LTR from a lentivirus. The 3′ LTR may be made self-inactivating by any method known in the art. In a preferred embodiment, the U3 element of the 3′ LTR contains a deletion of its enhancer sequence, preferably the TATA box, Sp1 and NF-kappa B sites. As a result of the self-inactivating 3′ LTR, the provirus that is integrated into the host genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the multiple-promoter RNAi expression cassettes of the present invention to cells of interest, including but not limited to gene-deleted adenovirus-transposon vectors that stably maintain virus-encoded transgenes in vivo through integration into host cells (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus; or mini-circle DNA vectors devoid of bacterial DNA sequences (see Chen, et al., Molecular Therapy. 8(3):495-500 (2003)). Mini-circle DNA as described in U.S. Patent Publication No. 2004/0214329 discloses vectors that provide for persistently high levels of nucleic acid transcription. The circular vectors are characterized by being devoid of expression-silencing bacterial sequences, and may include a unidirectional site-specific recombination product sequence in addition to an expression cassette.

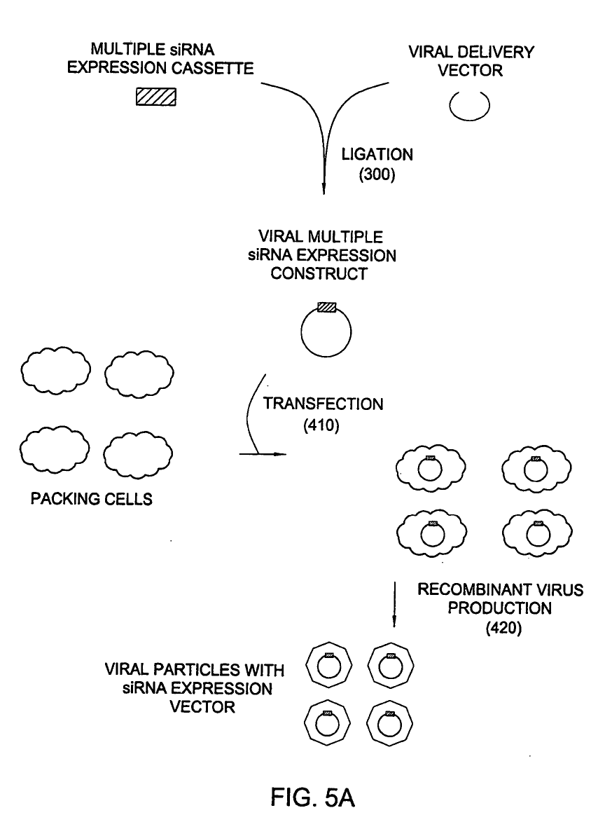

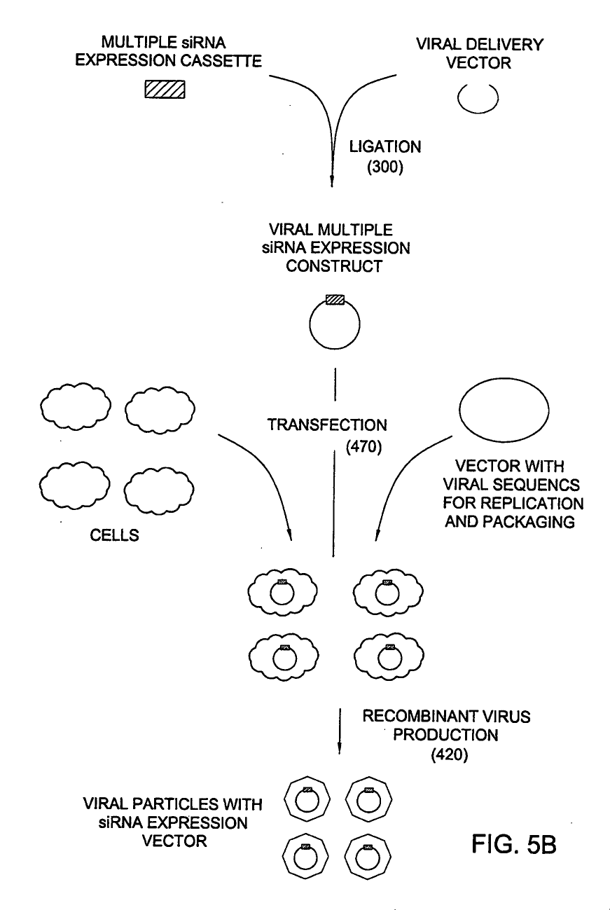

In step 400 of FIG. 1, the multi-RNAi expression construct is packaged into viral particles. Any method known in the art may be used to produce infectious viral particles whose genome comprises a copy of the viral multiple-RNAi expression construct. FIGS. 5A and 5B show alternative methods for packaging the multiple-RNAi expression constructs of the present invention into viral particles for delivery. The method in FIG. 5A utilizes packaging cells that stably express in trans the viral proteins that are required for the incorporation of the viral multiple-RNAi expression construct into viral particles, as well as other sequences necessary or preferred for a particular viral delivery system (for example, sequences needed for replication, structural proteins and viral assembly) and either viral-derived or artificial ligands for tissue entry. In FIG. 5A, a multiple-RNAi expression cassette is ligated to a viral delivery vector (step 300), and the resulting viral multiple-RNAi expression construct is used to transfect packaging cells (step 410). The packaging cells then replicate viral sequences, express viral proteins and package the viral multiple-RNAi expression constructs into infectious viral particles (step 420). The packaging cell line may be any cell line that is capable of expressing viral proteins, including but not limited to 293, HeLa, A549, PerC6, D17, MDCK, BHK, bing cherry, phoenix, Cf2Th, or any other line known to or developed by those skilled in the art. One packaging cell line is described, for example, in U.S. Pat. No. 6,218,181.

Alternatively, a cell line that does not stably express necessary viral proteins may be co-transfected with two or more constructs to achieve efficient production of functional particles. One of the constructs comprises the viral multiple-RNAi expression construct, and the other plasmid(s) comprises nucleic acids encoding the proteins necessary to allow the cells to produce functional virus (replication and packaging construct) as well as other helper functions. The method shown in FIG. 5B utilizes cells for packaging that do not stably express viral replication and packaging genes. In this case, the multiple-RNAi expression construct is ligated to the viral delivery vector (step 300) and then co-transfected with one or more vectors that express the viral sequences necessary for replication and production of infectious viral particles (step 430). The cells replicate viral sequences, express viral proteins and package the viral multiple-RNAi expression constructs into infectious viral particles (step 420).

After production in a packaging cell line, the viral particles containing the multiple-RNAi expression constructs are purified and quantified (titered). Purification strategies include density gradient centrifugation, or, preferably, column chromatographic methods. The selection of a viral or non-viral delivery method is based upon whether a particular cell type or tissue will be the target of the RNAi therapeutic, or whether treatment will be systemic, whether a persistent or transitory silencing effect is preferred, the mode of administration of the RNAi therapeutic, and the like.

In step 500 of FIG. 1, the multiple-RNAi expression construct is delivered to the cells to be treated. The multiple-RNAi expression construct of the present invention may be introduced into the cells in vitro or ex vivo and then subsequently placed into an animal or human to effect therapy, or administered directly to an organism, organ or cell by in vivo administration. Delivery by viral infection is a preferred method of delivery; however, any appropriate method of delivery of the multiple-RNAi expression construct may be employed. The vectors comprising the multiple-RNAi constructs can be administered to a mammalian host using any convenient protocol, where a number of different such protocols are known in the art.

The nucleic acids may be introduced into tissues or host cells by any number of routes, including viral infection, microinjection, or fusion of vesicles. Injection may also be used for intra-muscular administration, as described by Furth et al., Anal. Biochem. 115(205):365-368 (1992). The nucleic acids may be coated onto gold microparticles, and delivered intradermally by a particle bombardment device, or “gene gun” as described in the literature (see, for example, Tang et al., Nature. 356:152-154 (1992)), where gold microprojectiles are coated with the DNA, then bombarded into skin cells.

Another delivery method useful for the method of the present invention comprises the use of Cyclosert™ technology as described in U.S. Pat. No. 6,509,323 to Davis et.al. Cyclosert™ technology platform is based upon cup-shaped cyclic repeating molecules of glucose known as cyclodextrins. The “cup” of the cyclodextrin molecule can form “inclusion complexes” with other molecules, making it possible to combine the Cyclosert™ polymers with other moieties to enhance stability or to add targeting ligands. In addition, cyclodextrins generally have been found to be safe in humans (individual cyclodextrins currently enhance solubility in FDA-approved oral and IV drugs) and can be purchased in pharmaceutical grade on a large scale at low cost. These polymers are extremely water soluble, non-toxic and non-immunogenic at therapeutic doses, even when administered repeatedly. The polymers can easily be adapted to carry a wide range of small-molecule therapeutics at drug loadings that can be significantly higher than liposomes.

The vectors comprising the multiple-RNAi constructs can be formulated into preparations for injection or administration by dissolving, suspending or emulsifying them in an aqueous or nonaqueous solvent, such as oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In addition, the vectors comprising the multiple-RNAi constructs can be formulated into pharmaceutical compositions by combination with appropriate, pharmaceutically-acceptable carriers or diluents. In pharmaceutical dosage forms, the vectors comprising the multiple-RNAi constructs may be administered alone or in association or combination with other pharmaceutically active compounds. Those with skill in the art will appreciate readily that dose levels for vectors comprising the multiple-RNAi constructs will vary as a function of the nature of the delivery vehicle, the relative ease of transduction of the target cells, the expression level of the RNAi species in the target cells and the like.

The RNAi agents according to the present invention include those that can act upon autosomal dominant (AD) genotypes. Examples of AD diseases are adult polycystic kidney disease, Huntington chorea, and von Willebrand disease. AD diseases can be due to mutations in receptor proteins or structural proteins. In particular RNAi agents of this invention are directed to AD diseases that are associated with a SNP or a set of SNPs. For diseases in which a single gene has been found to be the causative agent, for example Distonia, Huntington's disease, or colon cancer, the multiple-RNAi construct of this invention acts to target several regions of the disease allele by utilizing allele specific SNP groups also known as haplotypes. This approach greatly increases the effectiveness of expression reduction because multiple targets on the same gene can be attacked. This minimizes chances that accessibility issues will impede the effectiveness of the RNAi agents of this present invention. For diseases in which multiple gene defects have been shown to be causative—for example Hirschsprung disease, osteoporosis, or glaucoma—the multiple promoter cassette of this present invention acts to target multiple genes simultaneously. Examples of possible disease correlations between the claimed SNPs with members of the genes of each classification are listed below for representative protein families.

Amylases

Amylase is responsible for endohydrolysis of 1,4-alpha-glucosidic linkages in oligosaccharides and polysaccharides. Variations in the amylase gene may be indicative of delayed maturation and of various amylase producing neoplasms and carcinomas.

Amyloid

The serum amyloid A (SAA) proteins comprise a family of vertebrate proteins that associate predominantly with high-density lipoproteins (HDL). The synthesis of certain members of the family is greatly increased in inflammation. Prolonged elevation of plasma SAA levels, as in chronic inflammation, results in a pathological condition, called amyloidosis, which affects the liver, kidney and spleen and which is characterized by the highly insoluble accumulation of SAA in these tissues. Amyloid selectively inhibits insulin-stimulated glucose utilization and glycogen deposition in muscle, while not affecting adipocyte glucose metabolism. Deposition of fibrillar amyloid proteins intraneuronally, as neurofibrillary tangles, extracellularly, as plaques and in blood vessels, is characteristic of both Alzheimer's disease and aged Down's syndrome. Amyloid deposition is also associated with type II diabetes mellitus.

Angiopoeitin

Members of the angiopoeitin/fibrinogen family have been shown to stimulate the generation of new blood vessels, inhibit the generation of new blood vessels, and perform several roles in blood clotting. This generation of new blood vessels, called angiogenesis, is also an essential step in tumor growth in order for the tumor to get the blood supply that it needs to expand. Variation in these genes may be increase the risk of any form of heart disease, numerous blood clotting disorders, stroke, hypertension and predisposition to tumor formation and metastasis. In particular, RNAi agents of this invention can reduce expression of variants and may be therapeutic as antihypertensive, chemotherapeutic, and anti-tumor agents.

Apoptosis-Related Proteins

Active cell suicide (apoptosis) is induced by events such as growth factor withdrawal and toxins. It is controlled by regulators, which have either an inhibitory effect on programmed cell death (anti-apoptotic) or block the protective effect of inhibitors (pro-apoptotic). Many viruses have found a way of countering defensive apoptosis by encoding their own anti-apoptosis genes preventing their target-cells from dying too soon. RNAi agents of this invention can be useful in targeting allelic variants of these genes.

Cadherin, Cyclin, Polymerase, Oncogenes, Histones, Kinases

Members of the cell division/cell cycle pathways such as cyclins, many transcription factors and kinases, DNA polymerases, histones, helicases and other oncogenes play a critical role in carcinogenesis where the uncontrolled proliferation of cells leads to tumor formation and eventually metastasis. Variation in these genes may be predictive of predisposition to any form of cancer, from increased risk of tumor formation to increased rate of metastasis. In particular, these variants can be targets for RNAi agents of this invention.

Colony-Stimulating Factor-Related Proteins

Granulocyte/macrophage colony-stimulating factors are cytokines that act in hematopoiesis by controlling the production, differentiation, and function of 2 related white cell populations of the blood, the granulocytes and the monocytes-macrophages. Autosomal dominant diseases related to this category of genes include Huntington's disease and osteoporosis. RNAi agents of this invention can target multiple allelic variants of these genes while not affecting the expression of the normal allele.

Complement-Related Proteins

Complement proteins are immune associated cytotoxic agents, acting in a chain reaction to exterminate target cells to that were primed with antibodies, by forming a membrane attack complex (MAC). The mechanism of killing is by opening pores in the target cell membrane. Variations in 20 complement genes or their inhibitors are associated with many autoimmune disorders. Modified serum levels of complement products cause edemas of various tissues, lupus (SLE), vasculitis, glomerulonephritis, renal failure, hemolytic anemia, thrombocytopenia, and arthritis. They interfere with mechanisms of ADCC (antibody dependent cell cytotoxicity), severely impair immune competence and reduce phagocytic ability. Variants of complement genes may also be indicative of type I diabetes mellitus, meningitis neurological disorders such as Nemaline myopathy, Neonatal hypotonia, muscular disorders such as congenital myopathy and other diseases. RNAi agents derived from multiple promoter cassettes of this invention are particularly effective in treatment of autosomal dominant diseases with multiple gene targets, such as lupus.

Other complex genetic diseases suited for methods of this invention include, but are not limited to osteoporosis, colon cancer, and glaucoma. These diseases have been associated with mutations in multiple genes and therefore effective targeting of multiple polymorphisms simultaneously would result in the decreased expression of multiple disease causing alleles.

Cytochrome

The respiratory chain is a key biochemical pathway which is essential to all aerobic cells. There are five different cytochromes involved in the chain. These are heme bound proteins which serve as electron carriers. Modifications in these genes may be predictive of ataxia, dementia and myopathic and neuropathic changes in muscles. RNAi agents of this invention can be used to reduce expression of disease causing allelic variants, while leaving the expression of normal alleles unaffected.

Kinesins

Kinesins are tubulin molecular motors that function to transport organelles within cells and to move chromosomes along microtubules during cell division. Modifications of these genes may be indicative of neurological disorders such as Pick disease of the brain, tuberous sclerosis.

G-protein Coupled Receptors

G-protein coupled receptors (also called R7G) are an extensive group of hormones, neurotransmitters, odorants and light receptors which transduce extracellular signals by interaction with guanine nucleotide-binding (G) proteins. Alterations in genes coding for G-coupled proteins may be involved in and indicative of a vast number of physiological conditions. These include blood pressure regulation, renal dysfunctions, male infertility, dopamine associated cognitive, emotional, and endocrine functions, hypercalcemia, chondrodysplasia and osteoporosis, pseudohypoparathyroidism, growth retardation and dwarfism.

In one aspect of this invention an RNAi agent can affect the expression of mutated genes related to autosomal dominant polycystic kidney disease (ADPKD). This disease is the most common inherited kidney disease, with over 600,000 cases in the U.S. alone. The disease is characterized by renal insufficiency leading to renal transplantation. Mutation screening in the major gene for ADPKD, the polycystic kidney disease type 1 (PKD1) gene, has often been incomplete because of multiple homologous copies of this gene elsewhere on chromosome 16. Mutations have been found co-segregating with ADPKD in a study of 16 families linked to PKD1 by haplotype analysis. Of these mutations, six were insertions/deletions, five nonsense mutations, and five missense mutations. In the PKD2-linked family, a missense mutation, R322Q was found. RNAi agents of the present invention can specifically decrease the expression of mutated genes leading to the polycystic kidney disease. RNA agents of the present invention can target multiple polymorphisms in the same gene offering multiple sites for selective repression of a mutant allele.

In another aspect of this invention, multiple-RNAi agent constructs can affect the expression of mutated genes related to autosomal dominant Dystonia. Dystonia is a neurological movement disorder characterized by involuntary muscle contractions, which force certain parts of the body into abnormal, sometimes painful, movements or postures. Dystonia can affect any part of the body including the arms and legs, trunk, neck, eyelids, face, or vocal cords. Dystonia can be caused by mutation in the DYT1 gene encoding torsin A, an ATP-binding protein. RNAi agents of this invention can specifically decreases the expression of mutated genes leading to Dystonia. While Dystonia is an example of a single gene/single mutation disease, multiple promoter cassettes of this invention can be used to target allele specific SNPs in the torsion A gene. These SNPs may not necessarily confer the disease phenotype, but be merely associated with the disease genotype, thus providing a target for the RNAi agent of this invention.

TABLE 1
Exemplary SNT sequences which may be targeted using ddRNAi

| SNT | Entrez Gene ID No. | Disease |
|---|---|---|
| MCKD1 | 174000 | Polycystic kidney |
| MCKD 2 | 10122 | Polycystic kidney |
| PSEN1 | 5663 | Alzheimer's |
| PSEN2 | 5664 | Alzheimer's |
| APOA1 | 335 | amyloidosis |
| FGA | 2243 | amyloidosis |
| LYZ | 4069 | amyloidosis |
| FGA | 2243 | Afibrinogenemia |
| FGB | 2244 | Afibrinogenemia |
| FGG | 2266 | Afibrinogenemia |
| TNF | 7124 | apoptosis |
| RB1 | 5925 | retinoblastoma |
| HD | 3064 | Huntingtons disease |
| FCGBP | 8857 | Lupus |
| SLEB3 | 64695 | Lupus |
| SLEB2 | 56179 | Lupus |
| PDCD1 | 5133 | Lupus |
| SLEV1 | 140652 | Lupus |
| SLEH1 | 170682 | Lupus |
| KCNA1 | 3736 | ataxia |
| MAPT | 4137 | Picks Disease |
| Dyt1 | 266606 | Dystonia |
| APC | 324 | Colon cancer |
| MSH2 | 4436 | Colon cancer |
| CLCN7 | 1186 | osteoporosis |
| COL1A1 | 1277 | osteoporosis |
| CALCR | 799 | osteoporosis |
| MYOC | 4653 | glaucoma |
| OPTN | 10133 | glaucoma |
| RET | 5979 | Hirschsprung disease |
| EDNRB | 1910 | Hirschsprung disease |

Other agents that are useful in conjunction with the present invention will be readily apparent to those of skill in the art.

### EXAMPLES

Methods and examples describing multiple RNA cassettes are described in U.S. patent application Ser. No. 11/072,592, entitled “Multiple Promoter Expression Cassettes for Simultaneous Delivery of RNAi Agents” by inventors Petrus W. Roelvink, David A. Suhy, and Alexander Kolykhalov, which is incorporated by reference herein.

### Example 1

Testing of shRNA Triple Promoter Constructs for Synergistic Control of Gene Expression

In order to observe the effect of a multiple-RNA construct targeted to a single gene, triple promoter cassettes of the type shown in FIG. 3C are generated with the following promoters; U6-9 in position A, U6-1 in position B, and U6-8 in position C. The promoters drive transcription of shRNA sequences targeting various positions in a test gene. Single and double promoter/shRNA constructs are created to measure the effect of one or two shRNA agents targeting a single gene. The single, double or triple RNAi constructs are co-transfected with luciferase-target gene reporter plasmids that contain all three regions of the target gene. Luciferase activity is measured 72 hours after co-transfection into Huh7 cells. shRNA specific for the one region of the target shows the appropriate inhibitory activity to luciferase reporter plasmid containing that region of the gene. No non-specific inhibition is observed when single or multiple shRNA specific for regions of the target gene that are not included in the reporter plasmid are expressed. As more shRNAs targeting different regions of the gene are added to the expression cassette, expression of the target gene is reduced.

All publications, patents and patent applications cited herein are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably with departing from the basic principles of the invention.

1. A method for affecting gene expression of one or more genes in a subject or cell culture, said method comprising:
administering to said subject or cell culture a genetic construct comprising at least one ddRNAi expression cassette which encodes an RNA molecule comprising one, two or multiple RNAi nucleotide sequences which are individually at least 90% identical to at least part of a nucleotide sequence comprising one or more single nucleotide genetic targets or derivatives, orthologs or homologs thereof and which delay, repress or otherwise reduce the expression of one or more single nucleotide genetic targets of a heterozygotic pair in said subject or cell culture while not affecting the expression of another allele of the heterozygotic pair.

2. A genetically modified cell comprising at least one ddRNAi expression cassette which encodes an RNA molecule comprising one, two or multiple RNAi nucleotide sequences which are individually at least 90% identical to at least part of a nucleotide sequence comprising one or more single nucleotide genetic targets or derivatives, orthologs or homologs thereof and which delay, repress or otherwise reduce the expression of one or more single nucleotide genetic targets of a heterozygotic pair in a subject or cell culture while not affecting the expression of another allele of the heterozygotic pair.
